(19) United States
(12) Patent Application Publication
(10) Pub. No.: US 2025/0174150 A1
(43) Pub. Date: May 29, 2025

(54) METHOD AND SYSTEM FOR DETECTING UNHEALTHY HABITS OF USING MOBILE TERMINAL, AND RELATED DEVICE

(71) Applicant: AAC Acoustic Technologies (Shanghai) Co., Ltd., Shanghai (CN)

(72) Inventor: Nian Xiao, Shanghai (CN)

(21) Appl. No.: 18/629,978

(22) Filed: Apr. 9, 2024

Related U.S. Application Data
(63) Continuation of application No. PCT/CN2023/133844, filed on Nov. 24, 2023.

(51) Int. Cl. G09B 19/00; G09G 5/10

(52) U.S. Cl. CPC G09B 19/00; A61B 3/113; A61B 5/163; G06F 3/012; G06F 3/013

(57) ABSTRACT

Disclosed are a method, system, computer device and computer-readable storage medium for detecting unhealthy habits of using a mobile terminal. The detection method includes: detecting a viewing distance between a user and the mobile terminal via a distance sensor of the mobile terminal; determining whether the viewing distance is less than a preset threshold, and, based on a determination that the viewing distance is less than the preset threshold, determining the current viewing distance as unhealthy and reminding the user to perform distance correction; and ending the reminding based on a determination that the user has performed the distance correction and the viewing distance is greater than the preset threshold. The present disclosure can remind the user to correct the viewing distance and attitude, thus reducing the adverse effects of the mobile terminal on the user's eyes and body.

### CROSS REFERENCE TO RELATED APPLICATIONS

The present application is a continuation of PCT Patent Application No. PCT/CN2023/133844, entitled “METHOD AND SYSTEM FOR DETECTING UNHEALTHY HABITS OF USING MOBILE TERMINAL, AND RELATED DEVICE,” filed on Nov. 24, 2023, which is incorporated by reference herein in its entirety

### TECHNICAL FIELD

Embodiments of the present disclosure relate to the technical field of information detection, and more particularly to a method and a system for detecting unhealthy habits of using a mobile terminal, and a related device.

### BACKGROUND

As mobile terminals such as mobile phones and tablet computers become more and more popular, more and more children have their own or use their family's mobile phones and tablet computers. However, many children experience health issues such as neck problems, torticollis, hunchback, and strabismus due to unhealthy habits in using mobile terminals. These health issues are increasingly prominent among adolescents. Under poor environmental lighting conditions, long-time viewing of electronic displays, too close viewing distances, or incorrect viewing attitudes are the main causes of problems such as myopia and hunchback.

However, current mobile phones and tablet computers do not have the function of protecting children's eyes and attitudes. How to discover children's unhealthy habits by detecting children's viewing distance and sitting attitude, and provide them with scientific and reasonable tips so as to prevent them from developing health problems has become an urgent problem to be solved.

Therefore, it is desirable to provide a new method and a system, for detecting unhealthy habits of using a mobile terminal, and a related device, to solve the above technical problems.

### SUMMARY

Embodiments of the present disclosure are intended to provide a method and a system for detecting unhealthy habits of using a mobile terminal, so as to reduce the adverse effect of mobile terminals on children.

In a first aspect, embodiments of the present disclosure provide a method for detecting unhealthy habits of using a mobile terminal. The detection method includes:
detecting a viewing distance between a user and the mobile terminal via a distance sensor of the mobile terminal;
determining whether the viewing distance is less than a preset threshold, and, based on a determination that the viewing distance is less than the preset threshold, determining the current viewing distance as unhealthy and reminding the user to perform distance correction; and
ending the reminding based on a determination that the user has performed the distance correction and the viewing distance is greater than the preset threshold.

As an improvement, the detection method further includes:
detecting a usage attitude of the user via an attitude sensor of the mobile terminal;
determining whether the usage attitude conforms to a preset standard attitude, and, based on a determination that the usage attitude does not conform to the preset standard attitude, determining the current usage attitude as unhealthy and reminding the user to perform attitude correction; and
ending the reminding based on a determination that the user has performed the attitude correction and the usage attitude conforms to the preset standard attitude.

As an improvement, the detection method further includes:
obtaining ambient light via a light sensor of the mobile terminal; and
reminding the user that the current light is not suitable for using the mobile terminal based on a determination that the ambient light exceeds a preset range.

As an improvement, a screen of the mobile terminal enters an eye protection mode based on a determination that a duration of the unhealthy viewing distance and the unhealthy usage attitude exceeds a first preset time.

As an improvement, the screen of the mobile terminal enters a protection mode based on a determination that the duration of the unhealthy viewing distance and the unhealthy usage attitude exceeds a second preset time.

As an improvement, the user is reminded of overuse of the eyes based on a determination that the user uses the mobile terminal beyond a preset usage time.

In a second aspect, embodiments of the present disclosure provide a system for detecting unhealthy habits of using a mobile terminal. The system includes:
a detection module, configured for detecting a viewing distance between a user and the mobile terminal via a distance sensor of the mobile terminal;
a determination module, configured for determining whether the viewing distance is less than a preset threshold, and, based on a determination that the viewing distance is less than the preset threshold, determining the current viewing distance as unhealthy and reminding the user to perform distance correction; and
an ending module, configured for ending the reminding based on a determination that the user has performed the distance correction and the viewing distance is greater than the preset threshold.

In a third aspect, embodiments of the present disclosure further provide a computer device. The computer device includes: a memory, a processor, and a program for detecting unhealthy habits of using a mobile terminal which is stored in the memory and executable by the processor, where the processor is configured to execute the program for detecting unhealthy habits of using the mobile terminal to implement the operations of the method for detecting unhealthy habits of using the mobile terminal as described in any one of the above embodiments.

In a fourth aspect, embodiments of the present disclosure further provide a computer-readable storage medium storing a program for detecting unhealthy habits of using a mobile terminal, where the program for detecting unhealthy habits of using the mobile terminal is executed by a processor to implement the operations of the method for detecting unhealthy habits of using the mobile terminal as described in any one of the above embodiments.

The embodiments of the present disclosure have the following beneficial effects: the viewing distance and attitude of the user during use of the mobile terminal are detected by using the sensor in the mobile terminal, and the user is reminded to correct the viewing distance and attitude, so as to reduce the adverse effects of the mobile terminal on the user's eyes and body.

### BRIEF DESCRIPTION OF THE DRAWINGS

In order to illustrate the technical solutions in the embodiments of the present disclosure more clearly, the drawings used in the description of the embodiments are briefly described below. It is apparent that the drawings in the following description show only some embodiments of the present disclosure, and other drawings may be obtained by those of ordinary skill in the art based on these drawings without any creative efforts. In the drawings,

FIG. 1 is a flowchart of a method for detecting unhealthy habits of using a mobile terminal according to an embodiment of the present disclosure;

FIG. 2 is a schematic structural diagram of a system for detecting unhealthy habits of using a mobile terminal according to an embodiment of the present disclosure; and

FIG. 3 is a schematic structural diagram of a computer device according to an embodiment of the present disclosure.

### DETAILED DESCRIPTION OF THE EMBODIMENTS

The technical solutions in the embodiments of the present disclosure will be described clearly and completely hereinafter in conjunction with the drawings in the embodiments of the present disclosure. Apparently, the described embodiments are only a part of the embodiments of the present disclosure, rather than all embodiments. Any other embodiments obtained by those of ordinary skill in the art based on the embodiments in the present disclosure without any creative efforts shall fall within the scope of protection of the present disclosure.

### First Embodiment

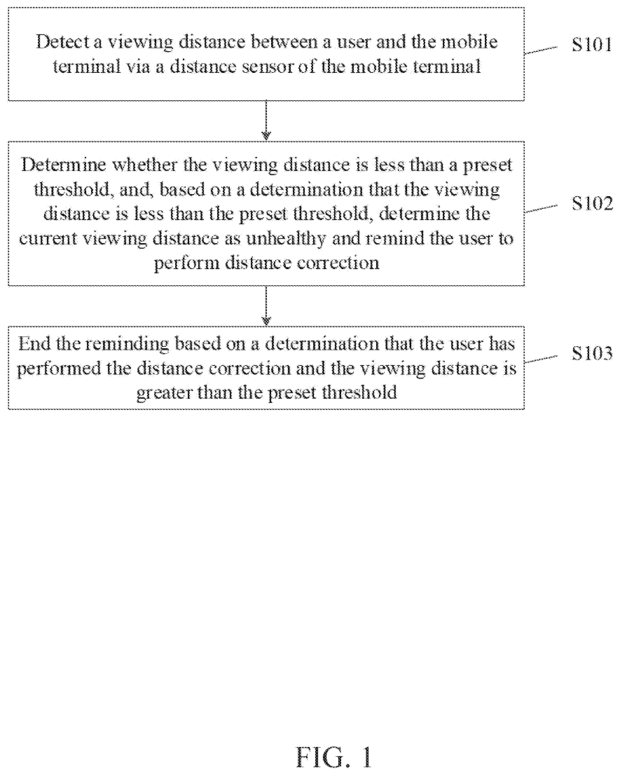

Referring to FIG. 1, embodiments of the present disclosure provide a method for detecting unhealthy habits of using a mobile terminal. The detection method includes the following operations S101 to S103.

In S101, a viewing distance between a user and the mobile terminal is detected via a distance sensor of the mobile terminal.

In this embodiment, the mobile terminal includes mobile phone, laptop, tablet computer, POS terminal, on-board computer, and so on. The viewing distance refers to the distance between the eyes and a screen of the mobile terminal.

In S102, it is determined whether the viewing distance is less than a preset threshold, and, based on a determination that the viewing distance is less than the preset threshold, the current viewing distance is determined as unhealthy and the user is reminded to perform distance correction.

In this embodiment, the preset threshold is set to 50-60 centimeters, which can reduce fatigue and discomfort of the eyes. The ways to remind the user include voice broadcast reminding and text reminding. For example, when a user is using a mobile phone, if the distance between the eyes and the phone screen is less than 50 centimeters, the phone broadcasts a piece of voice reminding the user to adopt the correct distance, and pops up a text window in the phone screen to remind the user for a period of time.

In this embodiment, the screen of the mobile terminal enters an eye protection mode if a duration of the unhealthy viewing distance and the unhealthy usage attitude exceeds a first preset time. The eye protection mode refers to a mode in which the blue light radiation caused by the screen is reduced and the screen light is adjusted to a more gentle warm light. This mode can effectively alleviate eye fatigue and protect vision.

In this embodiment, the screen of the mobile terminal enters a protection mode if the duration of the unhealthy viewing distance and the unhealthy usage attitude exceeds a second preset time. The protection mode refers to a mode in which an unclosable window is popped up in the mobile terminal to disturb the normal use of the mobile terminal and remind the user to stop the protection mode and continue using the mobile terminal only after the unhealthy body attitude and viewing distance are corrected and fall within a reasonable range.

For example, the first preset time is set to be less than the second preset time, which can limit the use of the mobile terminal by the user at different levels. If the user maintains the unhealthy viewing distance and the unhealthy usage attitude for a duration exceeding the first preset time, the screen of the mobile terminal enters the eye protection mode. Thus, it can protect the user's vision, and the change of light of the screen can also remind the user. If the user maintains the unhealthy viewing distance and the unhealthy usage attitude for a duration exceeding the second preset time, the screen of the mobile terminal imposes further restrictions on the basis of the eye protection mode, so that the user has to make corrections before continuing to use the mobile terminal.

In this embodiment, the user is reminded of overuse of the eyes if the user uses the terminal beyond a preset usage time.

In S103, the reminding is ended if the user has performed the distance correction and the viewing distance is greater than the preset threshold.

In this embodiment, the detection method further includes: detecting a usage attitude of the user via an attitude sensor of the mobile terminal; determining whether the usage attitude conforms to a preset standard attitude, and, based on a determination that the usage attitude does not conform to the preset standard attitude, determining the current usage attitude as unhealthy and reminding the user to perform attitude correction; and ending the reminding based on a determination that the user has performed the attitude correction and the usage attitude conforms to the preset standard attitude.

Specifically, the attitude sensor includes speed sensor, gyro sensor, and magnetic sensor. Alternatively, the attitude sensor may also be an optical sensor, such as a dtof module (direct time-of-flight sensing module), an itof module (indirect time-of-flight sensing module), and a vcsel structural light model (vertical-cavity surface-emitting laser structural light model), which can detect the user's tilt angle and orientation and further determine the user's attitude.

In this embodiment, the detection method further includes: obtaining ambient light via a light sensor of the mobile terminal; and reminding the user that the current light is not suitable for using the mobile terminal based on a determination that the ambient light exceeds a preset range.

In this embodiment, the detection method is integrated into the operating system or an application program of the mobile terminal, and the user can adjust various preset values (such as preset threshold, preset standard attitude, first preset time, and second preset time) in the operating system or the application program.

The embodiments of the present disclosure have the following beneficial effects: the viewing distance and attitude of the user during use of the mobile terminal are detected by using the sensor in the mobile terminal, and the user is reminded to correct the viewing distance and attitude, so as to reduce the adverse effects of the mobile terminal on the user's eyes and body.

### Second Embodiment

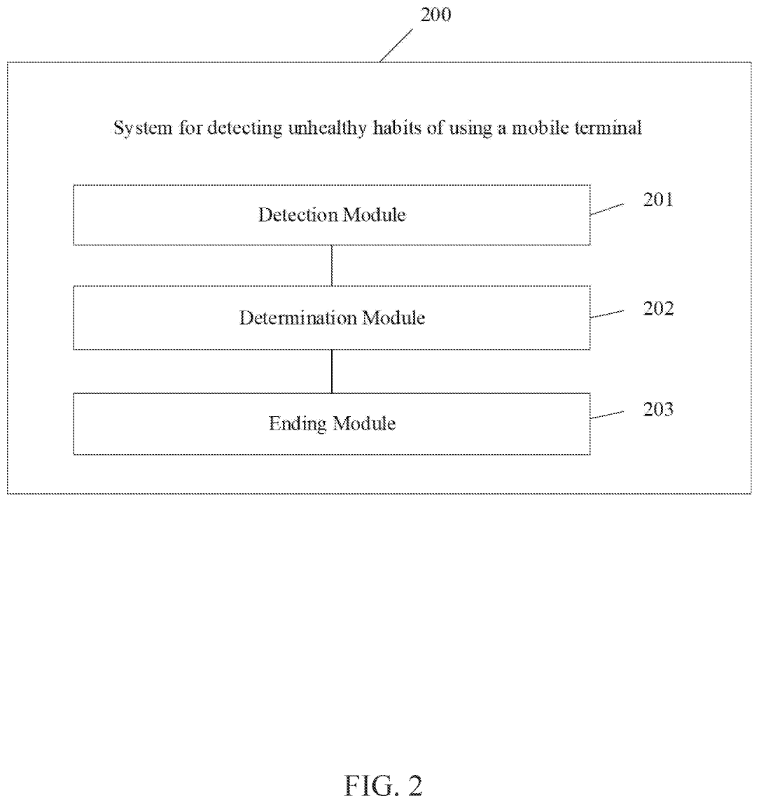

The embodiments of the present disclosure further provide a system for detecting unhealthy habits of using a mobile terminal. Referring to FIG. 2, FIG. 2 is a schematic structural diagram of the system 200 for detecting unhealthy habits of using a mobile terminal according to an embodiment of the present disclosure. The detection system includes: a detection module 201, a determination module 202, and an ending module 203.

The detection module 201 is configured for detecting a viewing distance between a user and the mobile terminal via a distance sensor of the mobile terminal.

In this embodiment, the mobile terminal includes mobile phone, laptop, tablet computer, POS terminal, on-board computer, and so on. The viewing distance refers to the distance between the eyes and a screen of the mobile terminal.

The determination module 202 is configured for determining whether the viewing distance is less than a preset threshold, and, based on a determination that the viewing distance is less than the preset threshold, determining the current viewing distance as unhealthy and reminding the user to perform distance correction.

In this embodiment, the preset threshold is set to 50-60 centimeters, which can reduce fatigue and discomfort of the eyes. The ways to remind the user include voice broadcast reminding and text reminding. For example, when a user is using a mobile phone, if the distance between the eyes and the phone screen is less than 50 centimeters, the phone broadcasts a piece of voice reminding the user to adopt the correct distance, and pops up a text window in the phone screen to remind the user.

In this embodiment, the screen of the mobile terminal enters an eye protection mode if a duration of the unhealthy viewing distance and the unhealthy usage attitude exceeds a first preset time. The eye protection mode refers to a mode in which the blue light radiation caused by the screen is reduced and the screen light is adjusted to a more gentle warm light. This mode can effectively alleviate eye fatigue and protect vision.

In this embodiment, the screen of the mobile terminal enters a protection mode if the duration of the unhealthy viewing distance and the unhealthy usage attitude exceeds a second preset time. The protection mode refers to a mode in which an unclosable window is popped up in the mobile terminal to disturb the normal use of the mobile terminal and remind the user to stop the protection mode and continue using the mobile terminal only after the unhealthy body attitude and viewing distance are corrected and fall within a reasonable range.

In this embodiment, the user is reminded of overuse of the eyes if the user uses the terminal beyond a preset usage time.

The ending module 203 is configured for ending the reminding based on a determination that the user has performed the distance correction and the viewing distance is greater than the preset threshold.

In this embodiment, the detection module 201 is further configured for detecting a usage attitude of the user via an attitude sensor of the mobile terminal; the determination module 202 is further configured for determining whether the usage attitude conforms to a preset standard attitude, and, based on a determination that the usage attitude does not conform to the preset standard attitude, determining the current usage attitude as unhealthy and reminding the user to perform attitude correction; and the ending module 203 is further configured for ending the reminding based on a determination that the user has performed the attitude correction and the usage attitude conforms to the preset standard attitude.

Specifically, the attitude sensor includes speed sensor, gyro sensor, and magnetic sensor. Alternatively, the attitude sensor may also be an optical sensor, such as a dtof module (direct time-of-flight sensing module), an itof module (indirect time-of-flight sensing module), and a vcsel structural light model (vertical-cavity surface-emitting laser structural light model), which can detect the user's tilt angle and orientation and further determine the user's attitude.

In this embodiment, the detection module 201 is further configured for obtaining ambient light via a light sensor of the mobile terminal; and the ending module 203 is further configured for reminding the user that the current light is not suitable for using the mobile terminal based on a determination that the ambient light exceeds a preset range.

The system 200 for detecting unhealthy habits of using a mobile terminal can implement the operations of the method for detecting unhealthy habits of using a mobile terminal in the above embodiment, and thus can achieve the same technical effects as described in the above embodiment, which will not be repeated here.

### Third Embodiment

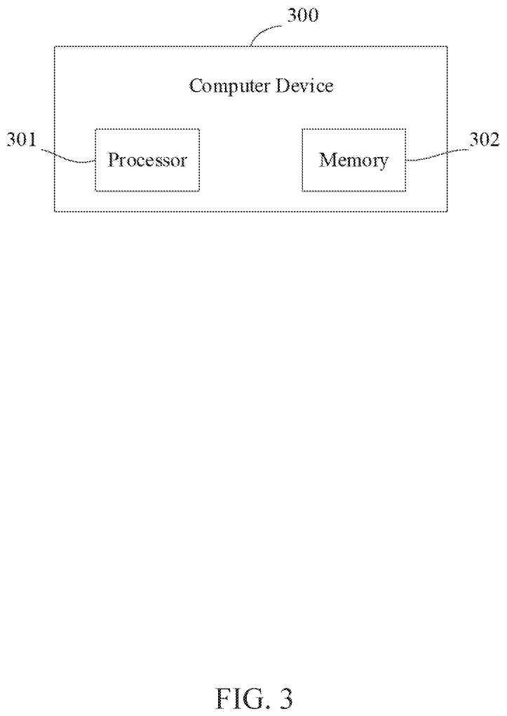

The embodiments of the present disclosure further provide a computer device. Referring to FIG. 3, FIG. 3 is a schematic structural diagram of a computer device according to an embodiment of the present disclosure. The computer device 300 includes: a memory 302, a processor 301, and a program for detecting unhealthy habits of using a mobile terminal which is stored in the memory 302 and executable by the processor 301.

The processor 301 is configured to execute the program, stored by the memory 302, for detecting unhealthy habits of using a mobile terminal to implement the operations S101-S103 of the method for detecting unhealthy habits of using a mobile terminal as provided by the embodiment of the present disclosure and as shown in FIG. 1.

In S101, a viewing distance between a user and the mobile terminal is detected via a distance sensor of the mobile terminal.

In S102, it is determined whether the viewing distance is less than a preset threshold, and, based on a determination that the viewing distance is less than the preset threshold, the current viewing distance is determined as unhealthy and the user is reminded to perform distance correction.

In S103, the reminding is ended if the user has performed the distance correction and the viewing distance is greater than the preset threshold.

The computer device 300 provided in the embodiment of the present disclosure can implement the operations of the method for detecting unhealthy habits of using a mobile terminal in the above embodiment, and thus can achieve the same technical effects as described in the above embodiment, which will not be repeated here.

### Fourth Embodiment

The embodiments of the present disclosure further provide a computer-readable storage medium storing a program for detecting unhealthy habits of using a mobile terminal. The program for detecting unhealthy habits of using a mobile terminal is executed by a processor to implement the procedures and operations of the method for detecting unhealthy habits of using a mobile terminal as provided by the embodiment of the present disclosure, and thus can achieve the same technical effects as described in the above embodiment, which will not be repeated here.

Those of ordinary skill in the art should understand that all or part of the procedures in the method of the above embodiment can be implemented by instructing relevant hardware through a computer program. The program may be stored in a computer-readable storage medium. When executed, the program may include the procedures of the embodiments of the above methods. The foregoing storage medium may be a magnetic disk, an optical disc, a read-only memory (ROM), or a random access memory (RAM).

It should be noted that, as used herein, the term “include”, “comprise” or any variant thereof is intended to encompass nonexclusive inclusion so that a process, method, article or device including a series of elements includes not only those listed elements but also other elements which have not been listed explicitly, or also includes an element(s) inherent to the process, method, article or device. Unless expressively limited otherwise, the statement “comprising (including) one . . . ” does not exclude the case that other similar components may exist in the process, method, article or device.

According to the foregoing descriptions of implementations, a person skilled in the art may clearly learn that the method according to the foregoing embodiments may be implemented by using software and a necessary general hardware platform, or certainly may be implemented by using hardware. However, in most cases, the former is a better implementation. Based on such understanding, the technical solutions in the present disclosure essentially or the part contributing to the conventional art may be implemented in the form of a software product. The computer software product is stored in a storage medium (such as ROM/RAM, a magnetic disk, or an optical disc), and includes several instructions for instructing a terminal device (which may be a mobile phone, a computer, a server, a network device, or the like) to perform the method described in the embodiments of the present disclosure.

The embodiments of the present disclosure have been described above in conjunction with the accompanying drawings, and the disclosed embodiments are only preferred embodiments of the present disclosure. However, the present disclosure is not limited to the above specific embodiments. The above specific embodiments are only illustrative rather than restrictive. Those of ordinary skill in the art can make various equivalent changes under the teaching of the present disclosure without departing from the spirit of the present disclosure and the scope of protection of the appended claims. All those equivalent changes fall within the scope of protection of the present disclosure.

What is claimed is:

1. A method for detecting unhealthy habits of using a mobile terminal, comprising:
detecting a viewing distance between a user and the mobile terminal via a distance sensor of the mobile terminal;
determining whether the viewing distance is less than a preset threshold, and, based on a determination that the viewing distance is less than the preset threshold, determining the current viewing distance as unhealthy and reminding the user to perform distance correction; and
ending the reminding based on a determination that the user has performed the distance correction and the viewing distance is greater than the preset threshold.

2. The method for detecting unhealthy habits of using a mobile terminal according to claim 1, further comprising:
detecting a usage attitude of the user via an attitude sensor of the mobile terminal;
determining whether the usage attitude conforms to a preset standard attitude, and, based on a determination that the usage attitude does not conform to the preset standard attitude, determining the current usage attitude as unhealthy and reminding the user to perform attitude correction; and
ending the reminding based on a determination that the user has performed the attitude correction and the usage attitude conforms to the preset standard attitude.

3. The method for detecting unhealthy habits of using a mobile terminal according to claim 1, further comprising:
obtaining ambient light via a light sensor of the mobile terminal; and
reminding the user that the current light is not suitable for using the mobile terminal based on a determination that the ambient light exceeds a preset range.

4. The method for detecting unhealthy habits of using a mobile terminal according to claim 2, wherein a screen of the mobile terminal enters an eye protection mode based on a determination that a duration of the unhealthy viewing distance and the unhealthy usage attitude exceeds a first preset time.

5. The method for detecting unhealthy habits of using a mobile terminal according to claim 2, wherein a screen of the mobile terminal enters a protection mode based on a determination that a duration of the unhealthy viewing distance and the unhealthy usage attitude exceeds a second preset time.

6. The method for detecting unhealthy habits of using a mobile terminal according to claim 1, wherein the user is reminded of overuse of the eyes based on a determination that the user uses the terminal beyond a preset usage time.

7. A system for detecting unhealthy habits of using a mobile terminal, comprising at least one processor and a storage medium, wherein the storage medium stores:
a detection module, configured for detecting a viewing distance between a user and the mobile terminal via a distance sensor of the mobile terminal;
a determination module, configured for determining whether the viewing distance is less than a preset threshold, and, based on a determination that the viewing distance is less than the preset threshold, determining the current viewing distance as unhealthy and reminding the user to perform distance correction; and
an ending module, configured for ending the reminding based on a determination that the user has performed the distance correction and the viewing distance is greater than the preset threshold.

8. A computer device, comprising: a memory, a processor, and a program for detecting unhealthy habits of using a mobile terminal which is stored in the memory and executable by the processor, wherein the processor is configured to execute the program for detecting unhealthy habits of using a mobile terminal to implement the operations of the method for detecting unhealthy habits of using a mobile terminal according to claim 1.

9. The computer device according to claim 8, wherein the processor is further configured to execute the program for detecting unhealthy habits of using a mobile terminal to implement the following operations:
detecting a usage attitude of the user via an attitude sensor of the mobile terminal;
determining whether the usage attitude conforms to a preset standard attitude, and, based on a determination that the usage attitude does not conform to the preset standard attitude, determining the current usage attitude as unhealthy and reminding the user to perform attitude correction; and
ending the reminding based on a determination that the user has performed the attitude correction and the usage attitude conforms to the preset standard attitude.

10. The computer device according to claim 8, wherein the processor is further configured to execute the program for detecting unhealthy habits of using a mobile terminal to implement the following operations:
obtaining ambient light via a light sensor of the mobile terminal; and
reminding the user that the current light is not suitable for using the mobile terminal based on a determination that the ambient light exceeds a preset range.

11. A computer-readable storage medium, wherein the computer-readable storage medium is configured for storing a program for detecting unhealthy habits of using a mobile terminal, wherein the program for detecting unhealthy habits of using a mobile terminal is executed by a processor to implement the operations of the method for detecting unhealthy habits of using a mobile terminal according to claim 1.

12. The computer-readable storage medium according to claim 11, wherein the program for detecting unhealthy habits of using a mobile terminal is executed to further implement the following operations:
detecting a usage attitude of the user via an attitude sensor of the mobile terminal;
determining whether the usage attitude conforms to a preset standard attitude, and, based on a determination that the usage attitude does not conform to the preset standard attitude, determining the current usage attitude as unhealthy and reminding the user to perform attitude correction; and
ending the reminding based on a determination that the user has performed the attitude correction and the usage attitude conforms to the preset standard attitude.

13. The computer-readable storage medium according to claim 11, wherein the program for detecting unhealthy habits of using a mobile terminal is executed to further implement the following operations:
obtaining ambient light via a light sensor of the mobile terminal; and
reminding the user that the current light is not suitable for using the mobile terminal based on a determination that the ambient light exceeds a preset range.
